(19) United States
(12) Patent Application Publication
(10) Pub. No.: US 2024/0159663 A1
(43) Pub. Date: May 16, 2024

(54) GAS DETECTION DEVICE

(71) Applicant: TOKYO GAS ENGINEERING SOLUTIONS CORPORATION, Minato-ku, Tokyo (JP)

(72) Inventors: Takeshi ABE, Minato-ku, Tokyo (JP); Tsuyoshi HARA, Minato-ku, Tokyo (JP)

(73) Assignee: TOKYO GAS ENGINEERING SOLUTIONS CORPORATION, Minato-ku, Tokyo (JP)

(21) Appl. No.: 18/550,394

(22) PCT Filed: Mar. 15, 2021
(86) PCT No.: PCT/JP2021/010369

§ 371 Date: Sep. 13, 2023

(51) Int. Cl. G01N 21/3504; G01N 33/00

(52) U.S. Cl. CPC G01N 21/3504; G01N 33/0062

(57) ABSTRACT

A gas detection device comprises a detection light emitting device which emits detection light used to detect a gas, a light receiving device which receives a reflected light of the detection light, and a control section which detects the gas on an optical path of the detection light from information on the reflected light received by the light receiving device. The gas detection device comprises a camera device which includes an optical axis generally aligning with an optical axis of the detection light emitted by the detection light emitting device, and a displaying device which displays a camera image taken by the camera device and displays an image showing an irradiated position by the detection light, overlapping the camera image.

### TECHNICAL FIELD

The present invention relates to a gas detection device.

### BACKGROUND ART

There is known a gas detection device which detects a gas such as methane gas by using infrared laser beam as detection light and measures a column density of the detected gas. Such a gas detection device emits infrared laser beam. The infrared laser beam is invisible light. Therefore, a user of the gas detection device cannot see and realize a position where the infrared laser beam is irradiated. Accordingly, there is known a gas detection device which emits visible laser beam such that an optical axis of the visible laser beam aligns with an optical axis of the infrared laser beam (for example, refer to Patent literature 1). Thereby, the position where the visible laser beam is irradiated, generally corresponds to the position where the infrared laser beam is irradiated. Thus, the user can know the position where the infrared laser beam is irradiated, based on the position where the visible laser beam is irradiated.

### PATENT LITERATURE

PTL 1: JP Patent 6630757 B

### SUMMARY OF INVENTION

### Problem to be Solved

As described above, the known gas detection device emits the visible laser beam such that the user can know the position where the infrared laser beam is irradiated. In this regard, the user may have difficulty to see and realize the position where the visible laser beam irradiates when the position where the visible laser beam irradiates is away from the user.

An object of the present invention is to provide a gas detection device which allows the user to successfully realize the position where the detection light is irradiated. Another object of the present invention is to provide a gas detection device which can unitarily manage information on a position where a gas to be detected leaks and an amount of the leaked gas to be detected, and a date of detecting leakage of the gas to be detected when the leakage of the gas to be detected is detected, and allow to appropriately perform maintenance with reference to the information.

### Means for Solving the Problem

A gas detection device according to the present invention, comprises a detection light emitting device which emits detection light used to detect a gas, a light receiving device which receives a reflected light of the detection light, and a control section which detects the gas on an optical path of the detection light from information on the reflected light received by the light receiving device.

The gas detection device comprises a camera device which includes an optical axis generally aligning with an optical axis of the detection light emitted by the detection light emitting device, and a displaying device which displays a camera image taken by the camera device and displays an image showing an irradiated position by the detection light, overlapping the camera image.

The gas detection device according to the present invention may comprise a guide light emitting device which emits guide light having an optical axis generally aligning with the optical axis of the detection light. In this case, the guide light is, for example, visible light.

Further, in the gas detection device according to the present invention, when the control section detects the gas on the optical path of the detection light, the control section may be configured to display information relating to the detected gas by the displaying device.

Further, in the gas detection device according to the present invention, the control section may be configured to display a detection result relating to the gas by the displaying device as the information relating to the gas.

Further, in the gas detection device according to the present invention, the control section may be configured to display a column density of the gas by the displaying device as the detection result relating to the gas.

Further, in the gas detection device according to the present invention, the detection light emitting device may emit infrared laser beam as the detection light.

Further in the gas detection device according to the present invention, the gas detection device may be configured to memorize information on the camera image taken when the gas is detected and the information relating to the detected gas in a storage device.

### Effects of Invention

With the gas detection device according to the present invention, the camera device takes images of the irradiated position where the detection light is irradiated and an area around the irradiated position. Therefore, the irradiated position image is displayed on the displaying device such that the irradiated position image is on the image of the irradiated position where the detection light is irradiated and the area around the irradiated position. Thus, the user of the gas detection device can successfully realize the irradiated position where the detection light is irradiated from the display by the displaying device.

With the present invention, when the leakage of the gas is detected by the gas detection device, the information on the positions where the gas leaks and the amounts of the leaked gas, and the dates of detecting the leakage of the gas is unitarily consolidated. Therefore, quality of the maintenance can be improved.

Elements of the invention are not limited to elements of embodiments and modified examples of the invention described with reference to the drawings. The other objects, features and accompanied advantages of the invention can be easily understood from the embodiments and the modified examples of the invention.

### BRIEF DESCRIPTION OF THE DRAWINGS

FIG. 1 is a view which shows a gas detection device and components thereof according to an embodiment of the present invention.

FIG. 2 is a view which shows the gas detection device and the components thereof according to the embodiment of the present invention.

FIG. 3 is a view which shows a display of a displaying device of the gas detection device according to the embodiment of the present invention.

FIG. 4 is a view which shows an optical system of the gas detection device according to the embodiment of the present invention.

FIG. 5 is a view which shows the gas detection device and a storage device according to the embodiment of the present invention.

FIG. 6 is a view similar to FIG. 3 and which shows the display when the gas detection device is turned on.

FIG. 7 is a view similar to FIG. 3 and which shows the display when a gas detection is being performed.

FIG. 8 is a view similar to FIG. 3 and which shows the display when a gas to be detected is detected.

FIG. 9 is a view which shows the display which displays camera image information and detected gas information acquired from the storage device.

### MODE FOR CARRYING OUT THE INVENTION

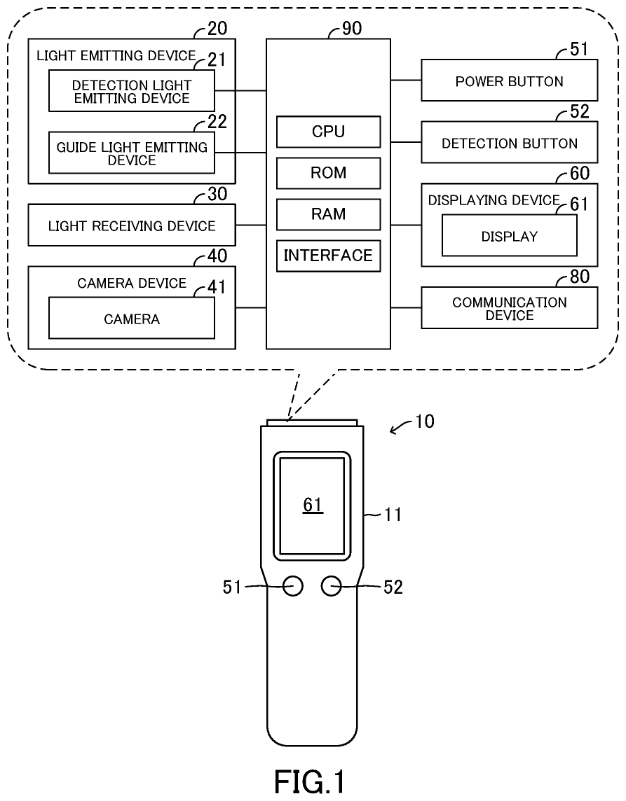

Below, a gas detection device according to an embodiment of the present invention will be described with reference to the drawings. The gas detection device 10 according to the embodiment of the present invention is shown in FIG. 1. The gas detection device 10 is a device which detects a predetermined gas (or a target gas to be detected).

As shown in FIG. 1, the gas detection device 10 includes a light emitting device 20, a light receiving device 30, a camera device 40, a power button 51, a detection button 52, a displaying device 60, a communication device 80, and an electronic control device 90 (hereinafter, will be referred to as “ECU 90”) as a control section.

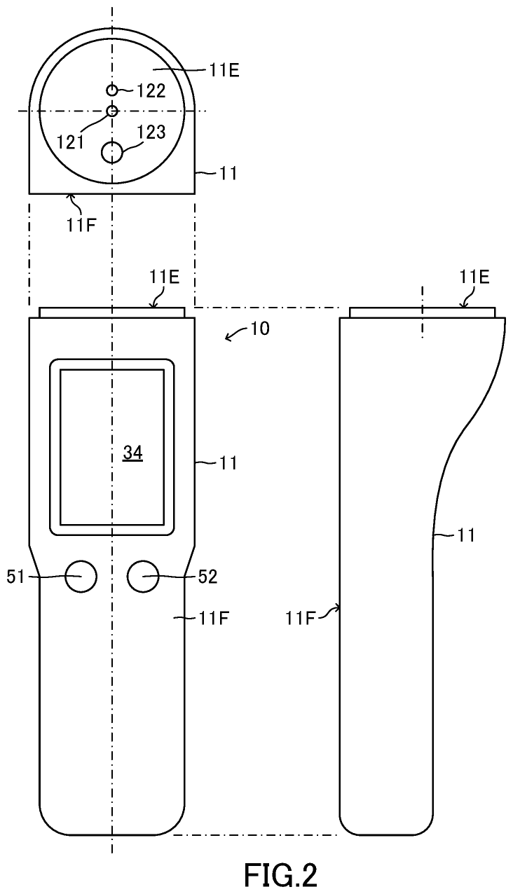

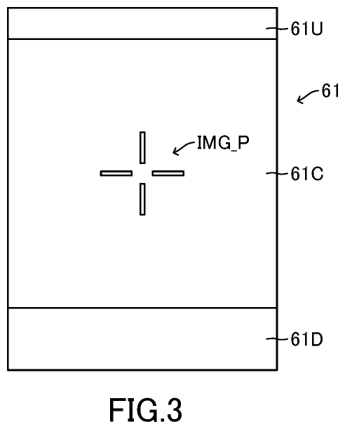

As shown in FIG. 2, a detection light emitting opening 121, a guide light emitting opening 122, and a camera lens opening 123 are formed on a top surface 11E of a body 11 of the gas detection device 10. In this embodiment, the guide light emitting opening 122 is formed on the side of a front surface 11F of the gas detection device 10 with respect to the detection light emitting opening 121, and the camera lens opening 123 is formed on the opposite side of the guide light emitting opening 122 with respect to the detection light emitting opening 121. Further, as shown in FIG. 3, the gas detection device 10 includes an optical system 70.

### <Light Emitting Device>

The light emitting device 20 is provided in the body 11 of the gas detection device 10 and includes a detection light emitting device 21 and a guide light emitting device 22.

### <Detection Light Emitting Device>

The detection light emitting device 21 emits detection light Ld used to detect the target gas (a gas to be detected by the gas detection device 10). In this embodiment, the target gas is methane gas, but may be hydrogen sulfide.

The detection light Ld is, for example, a frequency-modulated laser light. Frequency modulation of the laser light generates second harmonic signals, depending on a concentration of the gas. A wavelength (frequency) of the detection light Ld is preferably a wavelength that the detection light Ld is absorbed into the target gas, but the detection light Ld is not absorbed into a background gas (a gas such as water vapor other than the target gas). In this embodiment, the target gas is methane gas. Therefore, infrared laser beam having an oscillation wavelength range of 1.65 μm is used as the detection light Ld. Further, when the target gas is the hydrogen sulfide gas, the infrared laser beam having the oscillation wavelength range of 1.57 μm is used as the detection light Ld.

The detection light emitting device 21 is electrically connected to the ECU 90. The ECU 90 emits the detection light Ld from the detection light emitting device 21 and stops emitting the detection light Ld.

### <Guide Light Emitting Device>

As described above, in this embodiment, the target gas is the methane gas, and the detection light Ld is the invisible infrared laser beam. Therefore, a user of the gas detection device 10 cannot see and realize an irradiated position by the detection light Ld (a position where the detection light Ld is irradiated).

The guide light emitting device 22 emits guide light Lg which indicates the irradiated position by the detection light Ld (the position where the detection light Ld is irradiated). The guide light Lg is visible light such as red or green semiconductor laser beam. Further, the guide light emitting device 22 is positioned so as to emit the guide light Lg such that an optical axis of the guide light Lg aligns with an optical axis of the detection light Ld. Thus, an irradiated position by the guide light Lg (a position where the guide light Lg is irradiated) generally corresponds to the irradiated position by the detection light Ld. Therefore, the user of the gas detection device 10 can realize the irradiated position by the detection light Ld from the irradiated position by the guide light Lg.

The guide light emitting device 22 is electrically connected to the ECU 90. The ECU 90 emits the guide light Lg from the guide light emitting device 22 and stops emitting the guide light Lg.

In this regard, when a semiconductor laser oscillator is used in the detection light emitting device 21, it is preferred that a standard cell enclosing the target gas is prepared, and an operation temperature of the semiconductor laser oscillator is adjusted with reference to light absorption in the standard cell such that the oscillation wavelength of the detection light Ld corresponds to a center of an absorption line of the target gas.

### <Optical System>

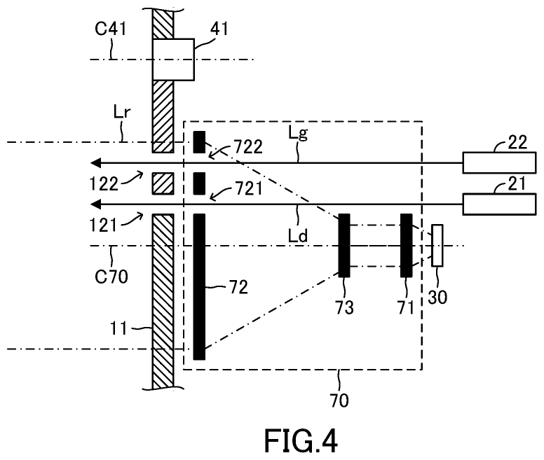

The optical system 70 is a device which focuses the detection light Ld reflected by an object (reflected light Lr). As shown in FIG. 4, the optical system 70 includes optical elements, i.e., a first optical element 71, a second optical element 72, and a third optical element 73. In this regard, in FIG. 4, a reference symbol C70 denotes an optical axis of the optical system 70.

The first optical element 71 is positioned closest to the light receiving device 30. The first optical element 71 is a light focusing element which focuses parallel lights on the light receiving device 30.

The second optical element 72 is positioned farthest from the light receiving device 30. The second optical element 72 has an opening radius greater than an opening radius of the first optical element 71. The second optical element 72 is a light focusing element which focuses parallel lights entering the optical system 70.

The third optical element 73 is positioned between the first optical element 71 and the second optical element 72. The third optical element 73 is a light diffusing element which diffuses the lights focused by the second optical element 72 to turn to the parallel lights. In this embodiment, the third optical element 73 is a single optical element, but may be an optical element configured by combining a plurality of optical elements.

In this embodiment, the second optical element 72 is positioned so as to shut off an optical path of the detection light Ld and an optical path of the guide light Lg, and the second optical element 72 is formed with a detection light passing hole 721 through which the detection light Ld passes and a guide light passing hole 722 through which the guide light Lg passes. The detection light emitting device 21 is positioned such that the emitted detection light Ld is emitted from the gas detection device 10 through the detection light passing hole 721 and the detection light emitting opening 121. The guide light emitting device 22 is positioned such that the emitted guide light Lg is emitted from the gas detection device 10 through the guide light passing hole 722 and the guide light emitting opening 122. Thus, the detection light Ld emitted from the detection light emitting device 21 is emitted from the gas detection device 10 through the detection light passing hole 721 and the detection light emitting opening 121, and the guide light Lg emitted from the guide light emitting device 22 is emitted from the gas detection device 10 through the guide light passing hole 722 and the guide light emitting opening 122.

In the gas detection device 10, the opening radius of the second optical element 72 is greater than the opening radius of the first optical element 71. Therefore, a greater amount of the reflected light Lr is gathered, the gathered reflected light Lr is focused, and the light having a greater intensity enters the light receiving device 30. Thus, information such as a column density of the target gas can be acquired with a high sensitivity. It should be noted that the column density of the target gas is an integral value of a concentration of the target gas on the optical path of the detection light Ld (and the reflected light Lr).

It should be noted that Fresnel lens may be used as at least one of the optical elements (preferably, all of the optical elements) of the optical system 70. Thereby, the optical system 70 can be rendered light and as a result, the gas detection device 10 can be rendered light.

### <Light Receiving Device>

The light receiving device 30 is a device which receives the reflected light Lr focused by the optical system 70 and outputs signals representing the intensity of the received reflected light Lr. The light receiving device 30 is configured, for example, by photoelectric conversion elements such as photodiodes and photo multipliers and drive detection circuits thereof. The light receiving device 30 may include optical filters such as band path filters, slits, and dispersing mechanisms.

The light receiving device 30 is electrically connected to the ECU 90. The light receiving device 30 sends received-light signals to the ECU 90. The ECU 90 acquires information on the target gas on the optical path of the detection light Ld, based on the received-light signals.

### <Camera Device>

The camera device 40 takes images of views outside of the gas detection device 10 and outputs the taken images (camera images). The camera device 40 includes a camera 41 such as a CCD camera. The camera device 40 is positioned so as to take the images of the views outside of the gas detection device 10 by the camera 41 through the camera lens opening 123.

Further, as shown in FIG. 4, the camera device 40 is positioned such that an optical axis C41 of the camera 41 aligns with the optical axis of the detection light Ld. Therefore, the camera device 40 takes the images of the views around the irradiated position by the detection light Ld by the camera 41.

The camera device 40 is electrically connected to the ECU 90. The camera device 40 sends the images taken by the camera 41 (or data on the images) to the ECU 90.

### <Displaying Device>

The displaying device 60 is a device which displays various images. The displaying device 60 includes a display 61 such as a liquid-crystal display. The displaying device 60 is positioned such that the user can see the display 61. In this embodiment, the displaying device 60 is positioned such that the display 61 exposes to outside of the gas detection device 10 at the front surface 11F of the body 11 of the gas detection device 10.

The displaying device 60 is electrically connected to the ECU 90. The ECU 90 causes the displaying device 60 to display various images.

### <Power Button and Detection Button>

The power button 51 is a device which is operated by the user to turn on and off the gas detection device 10. Further, the detection button 52 is a device which is operated by the user to start and stop a gas detection described later. The power button 51 and the detection button 52 are positioned so as to expose to the outside of the gas detection device 10 at the front surface 11F of the body 11 of the gas detection device 10.

The power button 51 and the detection button 52 are electrically connected to the ECU 90. When the power button 51 is operated by the user, the ECU 90 turns on or off the gas detection device 10. Further, when the detection button 52 is operated by the user, the ECU 90 starts or stops the gas detection.

### <Communication Device>

The communication device 80 is a device which performs communication with a storage devices 201 such as severs or clouds provided on an internet 200 via the internet 200. The communication device 80 is electrically connected to the ECU 90. Therefore, the ECU 90 can communicate with the storage device 201 provided on the internet 200 via the communication device 80.

### <Operations of Gas Detection Device>

Next, operations of the gas detection device 10 will be described.

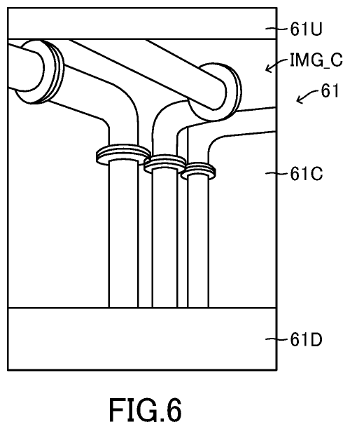

When the power button 51 is operated when the gas detection device 10 has been turned off, the ECU 90 turns on the gas detection device 10, starts taking the images by the camera device 40, and displays the images sent from the camera device 40 (camera images IMG_C) on a center area 61C of the display 61 and information such as a remaining amount of electricity of a battery installed in the gas detection device 10 on an upper area 61U of the display 61 as shown in FIG. 6.

When the detection button 52 is operated while the gas detection device 10 has been turned on, the ECU 90 starts a detection of the target gas (the gas detection).

When the ECU 90 starts the gas detection, the ECU 90 causes the detection light emitting device 21 to emit the detection light Ld. The detection light Ld emitted from the detection light emitting device 21 is emitted to the outside of the gas detection device 10 through the detection light passing hole 721 and then the detection light emitting opening 121. The detection light Ld emitted to the outside of the gas detection device 10 is irradiated on an object.

In addition, the ECU 90 causes the guide light emitting device 22 to emit the guide light Lg when the ECU 90 starts the gas detection. The guide light Lg emitted from the guide light emitting device 22 is emitted to the outside of the gas detection device 10 through the guide light passing hole 722 and then the guide light emitting opening 122. The guide light Lg emitted to the outside of the gas detection device 10 is irradiated on the object.

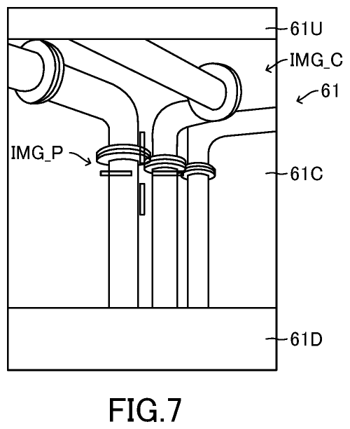

In addition, the ECU 90 displays an irradiated position image IMG_P on a camera image IMG_C in a center of the center area 61C of the display 61 as shown in FIG. 7 when the ECU 90 starts the gas detection. The irradiated position image IMG_P is an image which shows the irradiated position by the guide light Lg (i.e., the irradiated position by the detection light Ld).

The detection light Ld irradiated on the object is reflected by the object. The detection light Ld reflected by the object (the reflected light Lr) is received by the light receiving device 30. The light receiving device 30 sends the received-light signals representing the intensity of the received reflected light Lr to the ECU 90. The ECU 90 acquires information on light absorption by the target gas included in the reflected light Lr from the received-light signals and detects the target gas on the optical path of the detection light Ld from the acquired information. In particular, the ECU 90 detects “base wave signals having frequency equals to the modulated frequency of the detection light Ld” and “second harmonic signals having a twice frequency of the modulated frequency of the detection light Ld” from the received-light signals. A synchronous detection can be used to detect the base wave signals and the second harmonic signals. The ECU 90 detects the target gas in an atmosphere in which the background gas exists, based on a ratio of the base wave signal and the second harmonic signal.

When the ECU 90 detects the target gas, the ECU 90 acquires information on the detected target gas and displays an image showing the acquired information on the display 61.

The information on the target gas is, for example, a detection result relating to the target gas, and the detection result relating to the target gas is, for example, measured values of the target gas such as a column density of the target gas and a concentration of the target gas. In this regard, the detection result relating to the target gas may be a detection of the target gas.

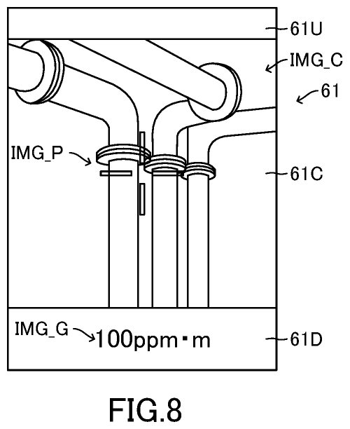

In this embodiment, when the ECU 90 detects the target gas, the ECU 90 acquires the column density of the detected target gas and displays an image IMG_G showing the acquired column density of the target gas in a lower area 61L of the display 61 as shown in FIG. 8.

With the gas detection device 10, the irradiated position image IMG_P is displayed on the display 61, overlapping the camera image IMG_C of the irradiated position by the detection light Ld and an area around the irradiated position by the detection light Ld. Therefore, the user of the gas detection device 10 can realize the irradiated position by the detection light Ld well from the image on the display 61 and can realize a position where the target gas exists well from the display 61 when the column density of the target gas is displayed on the display 61.

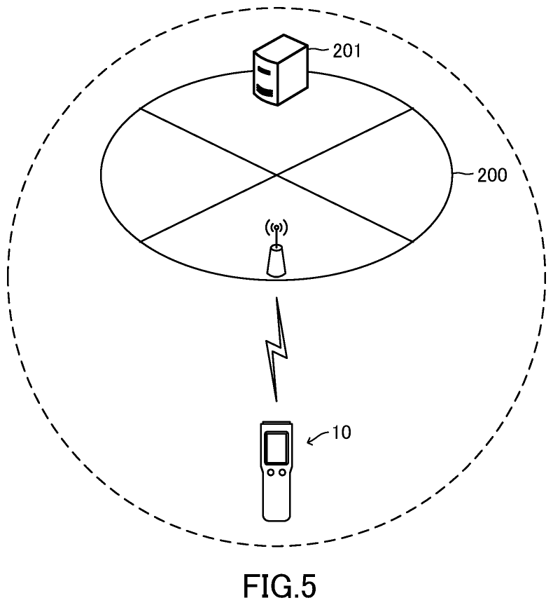

In addition, when the gas detection device 10 detects the target gas, the gas detection device 10 memorizes information on the camera image IMG_C taken when the gas detection device 10 detects the target gas (camera image information) and information relating to the detected target gas (target gas information) in the storage device 201 via the communication device 80 and the internet 200 (see FIG. 5).

In this embodiment, the target gas information includes the column density of the detected target gas, a place and/or a part where the target gas is detected, and a date and a point of time of detecting the target gas.

It should be noted that the gas detection device 10 may be provided with a button or a switch, and may be configured to memorize the camera image information and the target gas information in the storage device 201 when the user operates the button.

Later, the user can use the camera image information and the target gas information memorized in the storage device 201.

For example, the gas detection device 10 is configured to include a function to print a cord such as a QR code (registered trade mark) or a bar code for identifying the camera image information and the target gas information memorized in the storage device 201 on a paper or a sticker and a function to read the code and acquire the camera image information and the target gas information shown by the read cord from the storage device 201.

When the target gas is detected by the gas detection device 10, the user prints the code on the paper or the sticker and put the paper or the sticker on the part where the target gas is detected. Thereafter, the user causes the gas detection device 10 to read the code by the gas detection device 10 and acquires the camera image information and the target gas information shown by the read cord from the storage device 201.

It should be noted that the camera image information and the target gas information may be acquired by using a dedicated device including the function to read the code and acquire the camera image information and the target gas information shown by the read cord from the storage device 201.

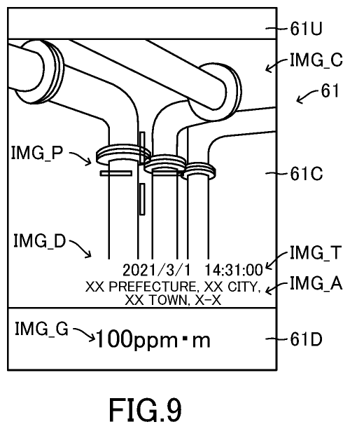

When the gas detection device 10 acquires the camera image information and the target gas information, the gas detection device 10 displays the camera image IMG_C shown by the acquired camera image information on the display 61 and displays the acquired target gas information on the display 61, overlapping the camera image IMG_C as shown in FIG. 9. In an example shown in FIG. 9, the gas detection device 10 displays the image IMG_G showing the column density of the target gas, an image IMG_D showing the date of the target gas being detected, an image IMG_T showing the point of time of the target gas being detected, and an image IMG_A showing an address of the place where the target gas being detected on the display 61.

With the gas detection device 10, maintenance persons can refer to the camera image information and the target gas information memorized in the storage device 201 when the maintenance persons carry out maintenance such as fixing of a leakage part of the gas. Thus, quality of the maintenance can be improved.

1. A gas detection device, comprising a detection light emitting device which emits detection light used to detect a gas, a light receiving device which receives a reflected light of the detection light, and a control section which detects the gas on an optical path of the detection light from information on the reflected light received by the light receiving device,
wherein the gas detection device comprises:
a camera device which includes an optical axis generally aligning with an optical axis of the detection light emitted by the detection light emitting device; and
a displaying device which displays a camera image taken by the camera device and displays an image showing an irradiated position by the detection light, overlapping the camera image.

2. The gas detection device as claimed in claim 1,
wherein the gas detection device comprises a guide light emitting device which emits guide light having an optical axis generally aligning with the optical axis of the detection light, and
wherein the guide light is visible light.

3. The gas detection device as claimed in claim 1, wherein when the control section detects the gas on the optical path of the detection light, the control section is configured to display information relating to the detected gas by the displaying device.

4. The gas detection device as claimed in claim 3, wherein the control section is configured to display a detection result relating to the gas by the displaying device as the information relating to the gas.

5. The gas detection device as claimed in claim 4, wherein the control section is configured to display a column density of the gas by the displaying device as the detection result relating to the gas.

6. The gas detection device as claimed in claim 1, wherein the detection light emitting device emits infrared laser beam as the detection light.

7. The gas detection device as claimed in claim 1, wherein the gas detection device is configured to memorize information on the camera image taken when the gas is detected and the information relating to the detected gas in a storage device.
